(19) United States
(12) Patent Application Publication
(10) Pub. No.: US 2026/0199193 A1
(43) Pub. Date: Jul. 16, 2026

(54) POWDERY SOLID COSMETIC

(71) Applicant: Shiseido Company, Ltd., Chuo-ku, Tokyo (JP)

(72) Inventor: Kento SUZUKI, Tokyo (JP)

(73) Assignee: Shiseido Company, Ltd., Chuo-ku, Tokyo (JP)

(21) Appl. No.: 19/133,381

(22) PCT Filed: Dec. 5, 2023
(86) PCT No.: PCT/JP2023/043397

§ 371 Date: May 28, 2025

(30) Foreign Application Priority Data
Dec. 28, 2022 (JP) 2022-212299

(51) Int. Cl. A61K 8/02; A61K 8/34

(52) U.S. Cl. CPC A61K 8/022; A61K 8/345

(57) ABSTRACT

A subject matter of the present disclosure is to provide a powdery solid cosmetic comprising a water-soluble agent, the cosmetic having excellent usability.

A powdery solid cosmetic of the present disclosure comprises (a) a water-soluble agent, (b) a usability-improving agent, and (c) a powder, wherein the (b) usability-improving agent comprises at least one selected from the group consisting of a polyhydric alcohol having a log P value of −1.5 or more, and a polyether having a number average molecular weight of 1000 or less.

### FIELD

The present disclosure relates to a powdery solid cosmetic.

### BACKGROUND

Powdery solid cosmetics containing a powder component, which cosmetics can be used for foundations and the like, are known.

PTL 1 discloses a powder-containing solid makeup cosmetic containing an oil of 33% by mass or more and a moisturizing agent of 3 to 15% by mass with respect to the total amount of the cosmetic.

PTL 2 discloses a solid powdery cosmetic comprising: (A) ethyl cellulose; (B) a monohydric liquid alcohol having a total number of carbon atoms of 18 to 24; (C) a powder; and (D) a liquid ester oil having a molecular weight of 1100 or less and containing one or more hydroxy groups; and the like.

### CITATION LIST

### Patent Literature

[PTL 1] Japanese Unexamined Patent Publication (Kokai) No. 2012-197241
[PTL 2] Japanese Unexamined Patent Publication (Kokai) No. 2019-104690

### SUMMARY

### Technical Problem

The present inventor attempted to obtain a powdery solid cosmetic comprising a water-soluble agent by the wet molding method. However, such a cometic sometimes had problems in its usability, including difficulty in removal of the cosmetic with a puff or the like.

The present inventor also attempted to obtain a powdery solid cosmetic comprising a water-soluble agent by the dry molding method. However, such a cometic sometimes had water droplets on its surface due to the effects of moisture in the atmosphere and the like (for example, the effect of hygroscopicity due to the deliquescence of the water-soluble agent). A cosmetic having water droplets on its surface also sometimes had problems in its usability, including difficulty in removal of the cosmetic with a puff or the like.

Accordingly, a subject matter of the present disclosure is to provide a powdery solid cosmetic comprising a water-soluble agent, the cosmetic having excellent usability.

### Solution to Problem

### <Aspect 1>

A powdery solid cosmetic comprising:
(a) a water-soluble agent;
(b) a usability-improving agent; and
(c) a powder;
wherein the (b) usability-improving agent comprises at least one selected from the group consisting of a polyhydric alcohol having a log P value of −1.5 or more, and a polyether having a number average molecular weight of 1000 or less.

### <Aspect 2>

The cosmetic according to aspect 1, wherein the (a) water-soluble agent comprises at least one selected from the group consisting of an ionic water-soluble agent, and a nonionic water-soluble agent having a log P value of less than 2.0.

### <Aspect 3>

The cosmetic according to aspect 1 or 2, wherein the content of the (a) water-soluble agent is 0.01% by mass or more with respect to the total amount of the cosmetic.

### <Aspect 4>

The cosmetic according to any one of aspects 1 to 3, wherein the (a) water-soluble agent comprises at least one selected from the group consisting of glycylglycine, potassium 4-methoxysalicylate, tranexamic acid or a salt thereof, dimethylpyrazolyl dimethylpyrimidine hydrochloride, hydroxyethyl imidazolidinone, and niacinamide.

### <Aspect 5>

The cosmetic according to any one of aspects 1 to 4, wherein the content of the (b) usability-improving agent is 0.1% by mass or more with respect to the total amount of the cosmetic.

### <Aspect 6>

The cosmetic according to any of aspects 1 to 5, wherein the (b) usability-improving agent comprises at least one selected from the group consisting of dipropylene glycol, 1,3-butylene glycol, and a polyethylene glycol having a number average molecular weight of 1000 or less.

### <Aspect 7>

A method of producing the cosmetic according to any one of aspects 1 to 6, wherein wet molding is carried out.

### <Aspect 8>

The production method according to aspect 8, wherein the wet molding uses water as a dispersion medium.

### <Aspect 9>

The method of producing the cosmetic according to any one of aspects 1 to 6, wherein dry molding is carried out.

### Advantageous Effects of Invention

According to the present disclosure, a powdery solid cosmetic comprising a water-soluble agent and having excellent usability can be provided.

### BRIEF DESCRIPTION OF DRAWINGS

FIG. 1 is a schematic diagram of a powdery solid cosmetic prepared with a constitution different from the constitution of the present disclosure, using the wet molding method. Panel (a) is a schematic diagram of the state of a slurry containing a water-soluble agent, a powder, and a dispersion medium, and Panel (b) is a schematic diagram of the powdery solid cosmetic after drying.

FIG. 2 is a schematic diagram of a powdery solid cosmetic prepared with a constitution of one embodiment of the present disclosure, using the wet molding method. Panel (a) is a schematic diagram of the state of a slurry containing a usability-improving agent, a water-soluble agent, a powder, and a dispersion medium, and Panel (b) is a schematic diagram of the powdery solid cosmetic after drying.

### DESCRIPTION OF EMBODIMENTS

Embodiments of the present disclosure are described below in detail with reference to drawings when necessary. The present disclosure is not limited to the following embodiments, and may be carried out with various variations within the scope of the main object of the invention.

The powdery solid cosmetic of the present disclosure comprises a predetermined usability-improving agent as well as a water-soluble agent and a powder.

Although the present invention is not limited by any principle, the action principle that allows the powdery solid cosmetic of the present disclosure to have excellent usability despite the inclusion of the water-soluble agent is considered to be as follows.

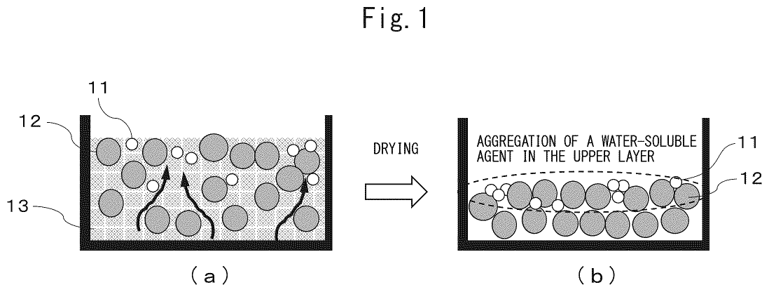

Production of a powdery solid cosmetic by, for example, the wet molding method involves the operation of removing the dispersion medium from the slurry by suction. In cases where a powdery solid cosmetic is produced using a water-soluble agent by this method, as illustrated in FIG. 1(a), water-soluble agent 11, dispersed in very small sizes in dispersion medium 13, is thought to move upward, passing between powder particles 12, due to an upward flow (arrows) generated by the suction of the dispersion medium. As a result, aggregation of the water-soluble agent 11 is thought to occur near the surface of the finally obtained powdery solid cosmetic, to adversely affect usability such as the removability with a puff. The aggregation of the water-soluble agent near the surface of the powdery solid cosmetic has been confirmed by, for example, results on the concentration gradient of the agent as obtained by quantitative analysis of the water-soluble agent along the thickness direction of the cosmetic.

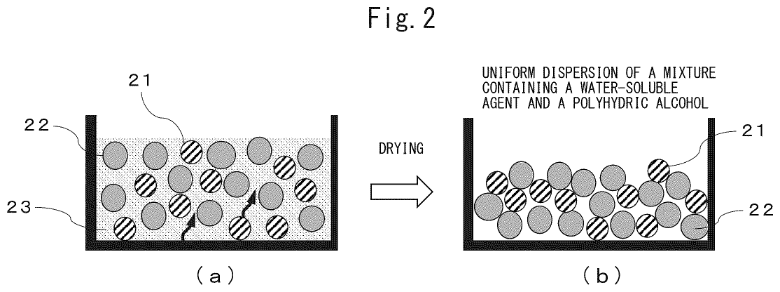

On the other hand, the powdery solid cosmetic of the present disclosure comprises a predetermined usability-improving agent as well as the water-soluble agent and the powder. Such a usability-improving agent is thought to produce an action similar to that of a surfactant since the agent not only has a hydrophilic performance, but also has a hydrophobic performance that is higher than those of glycerin and the like. Such a usability-improving agent is thought to integrate with a water-soluble agent present in the dispersion medium and, if present, an oil and the like, to form mixture 21 containing the water-soluble agent and the usability-improving agent as illustrated in FIG. 2(a). Further, since mixture 21 has a size larger than the water-soluble agent, its upward movement is thought to be inhibited by the powder and the like in the vicinity even when an upward flow is generated by the suction of the dispersion medium. It is therefore thought that, as a result, the water-soluble agent may be uniformly distributed in the cosmetic since the local aggregation of the agent may not occur at the surface of the finally obtained powdery solid cosmetic. Thus, the powdery solid cosmetic of the present disclosure is thought to enable reduction or suppression of an adverse effect on usability such as the removability with a puff.

Further, in cases where the powdery solid cosmetic is produced by the dry molding method or the like, even when, for example, the water-soluble agent tends to locally aggregate at the surface of the powdery solid cosmetic, the water-soluble agent is thought to be integrated with the usability-improving agent and, if present, an oil and the like, to be in the state of a mixture as described above in the powdery solid cosmetic of the present disclosure. Therefore, the water-soluble agent is thought to be protected such that the agent is incorporated by the usability-improving agent and the like. As a result, the water-soluble agent may not be placed in an exposed state at the surface of the powdery solid cosmetic, and hence the agent may be hardly affected by moisture and the like in the atmosphere (for example, the effect of hygroscopicity due to the deliquescence of the water-soluble agent). This is thought to be the reason why the problem of water droplets on the cosmetic surface, that is, the problem in the usability, could be improved.

Despite the fact that the powdery solid cosmetic of the present disclosure comprises a water-soluble agent, the cosmetic not only has excellent usability such as the removability with a puff, but is also capable of exhibiting an additional effect that can improve the finishing performance when the cosmetic is applied to the skin.

Typically, conventional powdery solid cosmetics have been prepared without using moisturizing agents. Since the powdery solid cosmetic of the present disclosure comprises a predetermined usability-improving agent that can function also as a moisturizing agent, the powdery solid cosmetic can also exhibit a moisturizing function. As a result, beauty effects for skin care and the like can also be improved compared to conventional powdery solid cosmetics.

The definitions of terms in the present disclosure are as follows

In the present disclosure, the term “usability” means the usability of a powdery solid cosmetic, and is intended to mean, for example, ease of taking a powdery solid cosmetic using a puff from a compact in which the cosmetic is filled (smoothness of the cosmetic), and applicability of the taken cosmetic to the skin (for example, the fact that the cosmetic can be lightly applied to the skin).

### <<Powdery Solid Cosmetic>>

The powdery solid cosmetic of the present disclosure comprises a powder, and may be in the form of the so-called pressed powder.

### <(a) Water-Soluble Agent>

The powdery solid cosmetic of the present disclosure comprises a water-soluble agent. A single type of water-soluble agent, or a combination of two or more types of water-soluble agents may be used. The water-soluble agent in the present disclosure does not include the later-described (b) usability-improving agent, and other polyhydric alcohols that are not included in the usability-improving agent.

The amount of the water-soluble agent included may be, for example, 0.01% by mass or more, 0.02% by mass or more, 0.03% by mass or more, 0.04% by mass or more, 0.05% by mass or more, 0.07% by mass or more, or 0.10% by mass or more, with respect to the total amount of the cosmetic. There is no upper limit value of the amount of the agent included, and the amount may be, for example, 6.0% by mass or less, 5.0% by mass or less, 3.0% by mass or less, 1.0% by mass or less, 0.70% by mass or less, 0.50% by mass or less, 0.40% by mass or less, 0.30% by mass or less, 0.20% by mass or less, or 0.10% by mass or less. The amount of the water-soluble agent included may be appropriately set within such a range depending on, for example, the required performance of the powdery solid cosmetic.

From the viewpoint of the interaction with the later-described usability-improving agent, usability of the cosmetic obtained, and the like, the water-soluble agent preferably includes at least one selected from the group consisting of an ionic water-soluble agent, and a nonionic water-soluble agent having a log P value of less than 2.0. These water-soluble agents may be referred to as “specific water-soluble agents”. The amount of the specific water-soluble agent(s) included may be, for example, 50% by mass or more, 60% by mass or more, 70% by mass or more, 80% by mass or more, or 90% by mass or more, and may be not more than 100% by mass, or less than 100% by mass, with respect to the total amount of the water-soluble agent in the cosmetic. A single type of specific water-soluble agent, or a combination of two or more types of specific water-soluble agents may be used.

The log P value of the nonionic water-soluble agent may be 1.8 or less, 1.5 or less, 1.3 or less, or 1.0 or less, and may be −4.0 or more, −3.8 or more, −3.5 or more, −3.3 or more, −3.0 or more, −2.7 or more, or −2.5 or more. The “log P value” herein is a parameter that represents the tendencies of partitioning of a substance into water and octanol, and is a value commonly used as an index to determine the degree of hydrophilicity or hydrophobicity of a substance. The smaller the log P value, the more hydrophilic the substance is meant to be. The larger the log P value, the more hydrophobic the substance is meant to be. The log P value may also be referred to as “octanol/water partition coefficient” according to the method of its calculation. The octanol/water partition coefficient can be determined by adding an agent into a flask containing octanol and an aqueous buffer solution at pH 7, shaking the flask, and then performing calculation according to the following Equation A based on the agent concentration in each phase:

$$Octanol / water partition coefficient = {Log}_{10} ( agent concentration in the octanol phase / \\ agent concentration in the aqueous phas e ) \qquad Equation A$$

Specific examples of such a specific water-soluble agent include alkoxysalicylic acid with a log P value of less than 2.0 (for example, 4-methoxysalicylic acid), B vitamins with a log P value of less than 2.0 or derivatives thereof, tranexamic acid (log P value: −2.0), L-ascorbic acid (log P value: 1.6), arbutin (log P value: −0.7), L-ascorbic acid glucoside (log P value: −3.1), 2-O-ethyl ascorbic acid (log P value: −1.5), glycylglycine (log P value: −2.3), 1-(2-hydroxyethyl)-2-imidazolidinone (log P value: −1.4), adenosine (log P value: −1.1), DIPY (dimethylpyrazolyl dimethylpyrimidine hydrochloride; log P value: 0.8), taurine (log P value: 1.4), trimethylglycine (log P value: 0.1), pyridoxine (log P value: −0.8), sodium pyrrolidonecarboxylate (log P value: −0.8), 1PP (1-piperidine propionic acid; log P value: −1.5), niacinamide (log P value: −0.4), trehalose (log P value: −1.7), xylitol (log P value: −2.5), sodium lactate (log P value: −0.7), urea (log P value: −2.1), hyaluronic acid (log P value: −7.4), and amino acids (log P values: −1.0 to −4.0) such as glutamic acid, alanine, and methionine, and derivatives thereof (including optical isomers). For example, among such agents, those that can be in the form of a salt, such as an alkoxysalicylate, for example, 4-methoxysalicylate; L-ascorbate; or tranexamic acid salt; may be used as the ionic water-soluble agent. Here, examples of the form of the salt include, but are not limited to, alkali metal salts (such as sodium salt, potassium salt, and lithium salt), alkaline earth metal salts (such as magnesium salt and calcium salt), ammonium salt, amino acid salt, sulfate, and hydrochloride.

Among the specific water-soluble agents, from the viewpoint of the interaction with the later-described usability-improving agent, usability of the cosmetic obtained, and the like, arbutin, pyridoxine, glycylglycine, 4-methoxysalicylate, tranexamic acid, dimethylpyrazolyl dimethylpyrimidine hydrochloride, hydroxyethyl imidazolidinone, and niacinamide are preferred. Glycylglycine, potassium 4-methoxysalicylate, tranexamic acid, dimethylpyrazolyl dimethylpyrimidine hydrochloride, hydroxyethyl imidazolidinone, and niacinamide are more preferred. Among such agents, those that can be in the form of a salt may also be suitably used.

### <(b) Usability-Improving Agent>

The powdery solid cosmetic of the present disclosure comprises, as a usability-improving agent, at least one selected from the group consisting of a polyhydric alcohol having a log P value of −1.5 or more, and a polyether having a number average molecular weight of 1000 or less. The term “usability-improving agent” in the present disclosure is intended to mean an agent whose inclusion in a powdery solid cosmetic enables improvement of usability such as the removability with a puff, compared to a powdery solid cosmetic comprising a water-soluble agent but not comprising a usability-improving agent. A single type of usability-improving agent, or a combination of two or more types of usability-improving agents may be used.

From the viewpoint of the interaction with the water-soluble agent, usability of the cosmetic obtained, and the like, the amount of the usability-improving agent included is preferably, for example, 0.1% by mass or more, 0.3% by mass or more, 0.5% by mass or more, 0.7% by mass or more, 1.0% by mass or more, 1.3% by mass or more, or 1.5% by mass or more, with respect to the total amount of the cosmetic. There is no upper limit value of the amount of the agent included, and the amount may be, for example, 10% by mass or less, 8.0% by mass or less, 5.0% by mass or less, or 3.0% by mass or less. The amount of the usability-improving agent included may be appropriately set within such a range, depending on the form of the powdery solid cosmetic and the like.

### (Polyhydric Alcohol)

As the usability-improving agent, a polyhydric alcohol (which may be simply referred to as “polyhydric alcohol”) having a log P value of −1.5 or more may be used. Here, in the present disclosure, the term “polyhydric alcohol” is intended to mean an organic compound having two or more alcoholic hydroxy groups in one molecule. The number of alcoholic hydroxy groups may also be defined as 10 or less, 8 or less, 6 or less, or 3 or less. It should be noted that the polyhydric alcohol in the present disclosure does not include the later-described polyether.

Examples of the polyhydric alcohol include dihydric alcohols and trihydric alcohols. Among these, from the viewpoint of the interaction with the water-soluble agent, usability of the cosmetic obtained, and the like, dihydric alcohols are preferred, and dihydric glycols are more preferred. A single type of polyhydric alcohol, or a combination of two or more types of polyhydric alcohols may be used.

Specific examples of such polyhydric alcohols include 1,3-butylene glycol (log P value: −0.4) and dipropylene glycol (log P value: −0.5). Among these, from the viewpoint of the interaction with the water-soluble agent, usability of the cosmetic obtained, and the like, dipropylene glycol is especially preferred. Glycerin, trehalose, and xylitol are not usability-improving agents in the present application since their log P values are −1.8, −1.7, and −2.5, respectively.

### (Polyether)

As the usability-improving agent, a polyether having a number average molecular weight of 1000 or less may be used. From the viewpoint of the interaction with the water-soluble agent, usability of the cosmetic obtained, and the like, the polyether is preferably a polyether in the form of a liquid (for example, in the form of a liquid at 25° C. under atmospheric pressure). A polyether is a chain polymer containing an ether bond (—C—O—C—) in the backbone, and has the structure of the following Formula 1:

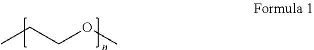

A polyether may also be referred to as a polyether compound or a polyether polyol. Specific examples of the polyether include polyethylene glycol, polypropylene glycol, polybutylene glycol, polyoxyethylene polyoxypropylene copolymers (for example, polyoxyethylene (9) polyoxypropylene (2) dimethyl ether, polyoxyethylene (14) polyoxypropylene (7) dimethyl ether, polyoxyethylene (36) polyoxypropylene (41) dimethyl ether, and polyoxyethylene (55) polyoxypropylene (28) dimethyl ether). Among these, from the viewpoint of the interaction with the water-soluble agent, usability of the cosmetic obtained, and the like, dihydric glycols are preferred. Polyethylene glycol, polypropylene glycol, and polybutylene glycol are more preferred. Polyethylene glycol is especially preferred. A single type of polyether, or a combination of two or more types of polyethers may be used.

From the viewpoint of the interaction with the water-soluble agent, usability of the cosmetic obtained, and the like, the number average molecular weight of the polyether is preferably 1000 or less, more preferably 800 or less, 700 or less, 600 or less, or 500 or less. There is no lower limit value of the molecular weight, and the molecular weight may be 100 or more, 150 or more, 200 or more, 250 or more, or 300 or more. Alternatively, the polyether is preferably a polyether in which the value of n in the above Formula 1 is 20 or less, 17 or less, 15 or less, 13 or less, or 10 or less. There is no lower limit of the value of n, and n may be 2 or more, 3 or more, 4 or more, or 5 or more. Preferably, the polyether has such a number average molecular weight and/or such a value of n, and is a compound in the form of a liquid at 25° C. under atmospheric pressure. Here, the term “number average molecular weight” is intended to mean the number average molecular weight in terms of polystyrene in gel permeation chromatography (GPC) measurement.

### <(c) Powder>

The powder used in the powdery solid cosmetic of the present disclosure is not limited. For example, a spherical or nonspherical powder commonly used in the art may be used. A single type of powder, or a combination of two or more types of powders may be used. The powdery solid cosmetic may comprise, as the powder, only a spherical powder or only a nonspherical powder. Alternatively, the powdery solid cosmetic may comprise both a spherical powder and a nonspherical powder.

In some embodiments, the powdery solid cosmetic of the present disclosure comprises a spherical powder. The size of the spherical powder is not limited. For example, the average particle size of the spherical powder may be 0.01 μm or more, 0.05 μm or more, 0.1 μm or more, 0.5 μm or more, 1.0 μm or more, 3.0 μm or more, or 5.0 μm or more, and may be 50 μm or less, 30 μm or less, 20 μm or less, 15 μm or less, or 10 μm or less. A single type of spherical powder, or a combination of two or more types of spherical powders, having an average particle size(s) within such a range may be used.

The average particle size may be defined as the average diameter of the powder (particles) optically measured by the dynamic light scattering method based on the assumption that the powder shape is spherical.

In some embodiments, the powdery solid cosmetic of the present disclosure comprises a nonspherical powder. The term “nonspherical powder” is intended to mean a powder not included in the above-described spherical powder, and it may be defined based on, for example, its aspect ratio. The aspect ratio of the nonspherical powder may be 1.2 or more, 1.5 or more, 2.0 or more, 2.5 or more, 3.0 or more, 5.0 or more, 7.0 or more, 10 or more, 15 or more, 20 or more, 25 or more, or 30 or more, and may be 200 or less, 170 or less, 150 or less, 120 or less, 100 or less, 70 or less, or 50 or less. A single type of nonspherical powder, or a combination of two or more types of nonspherical powders, having an aspect ratio within such a range may be used.

The aspect ratio can be calculated, for example, by randomly extracting 10 or more (for example, 100) powder particles under the microscope, measuring each powder particle in terms of the length in the long direction (plane direction) and the length in the short direction (thickness direction), and then dividing the length in the long direction by the length in the short direction (that is, the length in the long direction/the length in the short direction). Regarding the length in the long direction, the longest length is selected. Regarding the length in the short direction, the shortest length is selected. The aspect ratio may be the average of the aspect ratios of 10 or more (for example, 100) random powder particles.

The amount of the powder included is not limited, and may be, for example, 30% by mass or more, 40% by mass or more, 50% by mass or more, 55% by mass or more, 60% by mass or more, 65% by mass or more, 70% by mass or more, 75% by mass or more, 80% by mass or more, or 85% by mass or more, and may be 95% by mass or less, 93% by mass or less, 90% by mass or less, 87% by mass or less, 85% by mass or less, 83% by mass or less, or 80% by mass or less, with respect to the total amount of the cosmetic. The powder may be appropriately included in the cosmetic within such a range.

The type of the powder is not limited. For example, an inorganic powder and/or organic powder may be used. Each of the inorganic powder and the organic powder used may be a single type of powder or a combination of two or more types of powders.

Examples of a component constituting the inorganic powder include talc, kaolin, mica (such as sericite, white mica, phlogopite, synthetic mica, synthetic phlogopite iron, lepidolite, and biotite), calcined talc, calcined mica (such as calcined sericite, calcined white mica, and calcined phlogopite), vermiculite, magnesium carbonate, calcium carbonate, aluminum silicate, barium silicate, calcium silicate, magnesium silicate, strontium silicate, Ca/Al borosilicate, tungstic acid metal salts, magnesium, silica, alumina, zeolite, aluminum hydroxide, barium sulfate, calcined calcium sulfate (calcined gypsum), calcium phosphate, fluorapatite, hydroxyapatite, ceramic powders, metal soaps (such as zinc myristate, calcium palmitate, and aluminum stearate), boron nitride, photochromic titanium oxide (titanium dioxide sintered with iron oxide), and reduced zinc oxide. In some cases, such powders are included as pigments other than the following coloring pigments, for the purpose of increasing the amount of the cosmetic or reinforcing the cosmetic. Powders used for such purposes may be referred to as extender pigments.

Further, as the inorganic powder, for example, an inorganic pigment (which may be referred to as “inorganic coloring pigment”) may also be used. Examples of the inorganic pigment include inorganic white pigments (such as titanium dioxide and zinc oxide), inorganic red pigments (such as iron oxide (colcothar) and iron titanate), inorganic brown pigments (such as γ-iron oxide), inorganic yellow pigments (such as yellow iron oxide and loess), inorganic black pigments (such as black iron oxide and lower titanium oxides), inorganic violet pigments (such as mango violet and cobalt violet), inorganic green pigments (such as chromium oxide, chromium hydroxide, and cobalt titanate), and inorganic blue pigments (such as ultramarine blue and Prussian blue). Other examples of the inorganic pigment include photoluminescent powders. Examples of such photoluminescent powders include pearl pigments (such as bismuth oxychloride; fish scale flakes; mica titanium; iron oxide-coated mica titanium; lower titanium oxide-coated mica titanium; photochromic mica titanium; pearl pigments in which talc, glass, synthetic fluorophlogopite, silica, bismuth oxychloride, or the like is used instead of mica as a substrate; pearl pigments coated with a coating material containing titanium oxide, and also containing lower titanium oxide, colored titanium oxide, iron oxide, alumina, silica, zirconia, zinc oxide, cobalt oxide, aluminum, or the like; and functional pearl pigments, for example, pearl pigments whose surface is coated with resin particles, pearl pigments whose surface is coated with aluminum hydroxide particles, pearl pigments whose surface is coated with zinc oxide particles, and pearl pigments whose surface is coated with barium sulfate particles); and metal powder pigments (such as aluminum powders and copper powders).

Examples of a component constituting the organic powder include silicone elastomers; silicone; silicone resin-coated silicone elastomers; polyamide resins (such as nylon); polyolefin resins (such as polyethylene); polymethyl methacrylate; polystyrene; styrene-acrylic acid copolymer resins; benzoguanamine resins; fluorocarbon resins (such as polytetrafluoroethylene); starches (such as starch octenylsuccinate Al); crosspolymers such as (HDI/trimethylol hexyllactone) crosspolymers, (diphenyl dimethicone/vinyl diphenyl dimethicone/silsesquioxane) crosspolymers, and (IPDI/poly(1,4-butanediol)-14) crosspolymers; and cellulose.

Further, as the organic powder, an organic pigment (which may be referred to as “organic coloring pigment”) and/or a dye may also be used. Examples of the organic pigment include organic pigments such as zirconium, barium, or aluminum lakes, specifically, for example, organic pigments such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401, and Blue No. 404; and Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, and Blue No. 1.

Examples of the dye include natural dyes such as chlorophyll and β-carotene.

The powder used in the powdery solid cosmetic of the present disclosure may or may not be treated with a surface treatment agent. For example, in cases where a powder treated with a surface treatment agent such as carboxydecyl trisiloxane, dimethicone, silicone, myristic acid, stearic acid, dextrin palmitate, or an acrylic-silicone-based graft copolymer is used, the ratio of the treated powder to the entire powder may be 100% or less, 80% or less, 50% or less, 30% or less, 15% or less, 10% or less, 5% or less, 1% or less, 0.5% or less, 0.1% or less, 0.05% or less, or 0.01% or less, and may be 0.01% or more, 0.1% or more, 1% or more, 5% or more, 10% or more, 20% or more, 30% or more, 40% or more, or 50% or more.

### <Optional Components>

The powdery solid cosmetic of the present disclosure may comprise various components as required, as long as the effect of the present disclosure is not adversely affected. Examples of such optional components include components other than those described above that are used in cosmetic products and pharmaceutical products. Specific examples of the optional components include surfactants, water-soluble polymers, oil-soluble polymers, thickeners, film-forming agents, UV absorbers, discoloration inhibitors, sequestering agents, antiseptics, moisturizing agents (such as glycerin), polymer emulsions, pH adjustors, antiseptics, deodorants, microbicides, antioxidants, antioxidant aids, excipients, perfumes, color materials (such as pigments and dyes other than those described above, and colorants), oils, and water. A single type of optional component, or a combination of two or more types of optional components may be used.

In some embodiments, the powdery solid cosmetic of the present disclosure comprises an oil. The amount of the oil included may be, for example, 1.0% by mass or more, 3.0% by mass or more, 5.0% by mass or more, more than 5.0% by mass, 6.0% by mass or more, 8.0% by mass or more, or 10% by mass or more. There is no upper limit value of the amount of the oil included, and the amount may be, for example, 20% by mass or less, 17% by mass or less, 15% by mass or less, 13% by mass or less, or 10% by mass or less.

The term “oil” in the present disclosure refers to an oil commonly used in powdery solid cosmetics, and includes liquid oils and fats, solid oils and fats, waxes, hydrocarbon oils, silicone oils, and polar oils, but does not include the usability-improving agent described above. On the other hand, organic UV absorbers may be regarded as oils. A single type of oil, or a combination of two or more types of oils may be used.

### <<Use of Powdery Solid Cosmetic>>

The use of the powdery solid cosmetic of the present disclosure is not limited. Examples of the use include foundations (such as powdery foundations), eye shadows, cheek colors, body powders, perfume powders, baby powders, deodorant powders, and face powders.

### <<Method of Producing Powdery Solid Cosmetic>>

The method of producing the powdery solid cosmetic of the present disclosure is not limited, and a known method may be employed for the production. Examples of such a production method include the dry molding method and the wet molding method. In the method of producing the powdery solid cosmetic, the above-described various components may be used in the same manner.

In some embodiments, the powdery solid cosmetic of the present disclosure may be produced by the wet molding method. Since the powdery solid cosmetic of the present disclosure can be produced using a predetermined usability-improving agent capable of producing a surfactant-like action, the usability-improving agent is thought to integrate with the water-soluble agent in the dispersion medium and, if present, an oil and the like, to form a mixture as shown in FIG. 2. Thus, the formation of this mixture is thought to reduce or suppress movement of the water-soluble agent to the surface. From the viewpoint of the ability to form such a mixture, and the reduction or suppression of the movement of the water-soluble agent to the cosmetic surface as a result, it is preferred not to use the water-soluble agent as it is in the solid state. Instead, it is preferred to dissolve, before use, the water-soluble agent in a solvent in which the agent is soluble (such as water), and/or to use water as the dispersion medium for the slurry in the wet molding method so as to dissolve the water-soluble agent with the water contained in the dispersion medium. The content of water in the dispersion medium may be 50% by mass or more, 60% by mass or more, 70% by mass or more, 80% by mass or more, or 90% by mass or more, and may be not more than 100% by mass or less than 100% by mass, with respect to the total amount of the dispersion medium. The dispersion medium may also contain a component other than water, and examples of such a component include lower alcohols such as ethanol.

### EXAMPLES

Examples are described below to illustrate the present invention in more detail. However, the present invention is not limited thereto. Unless otherwise specified, each included amount is expressed in % by mass. “%” in Table 1 is intended to mean “% by mass”. The evaluation methods described in Examples may be carried out not only for the cosmetics described in Examples, but also for cosmetics comprising the above-described components.

### Examples 1 to 7 and Comparative Examples 1 to 7

Powdery solid cosmetics obtained according to the formulations shown in Table 1 by the production methods described below were subjected to the following evaluations. The results are shown in Table 1. Here, the powdery solid cosmetics correspond to pressed powder type foundations.

### <Evaluation Methods>

### (Usability Test 1: Removability of Cosmetic)

Overall evaluation of the usability was carried out by a panel of five experts. Regarding the evaluation method, usability at the time of collecting the cosmetic using a foundation puff was evaluated according to the following standard in terms of the ease of removing the cosmetic using the puff.
A: An appropriate amount of the cosmetic could be collected.
B: A small amount of the cosmetic could be collected.
C: The cosmetic could not be collected at all.

### (Usability Test 2: Applicability of Cosmetic to Skin)

Overall evaluation of the usability was carried out by a panel of five experts. Regarding the evaluation method, the cosmetic was collected using a foundation puff, and usability at the time of application of the cosmetic to the face was evaluated according to the following standard in terms of the smoothness during the application of the cosmetic.
A: Very smooth.
B: Slightly smooth.
C: Not very smooth.

### (Finishing Performance Test 1: Uniformity of Finishing)

Overall evaluation of the finishing performance was carried out by a panel of five experts. Regarding the evaluation method, the cosmetic was collected using a foundation puff, and the finishing performance at the time of application of the cosmetic to the face was evaluated according to the following standard in terms of the uniformity of finishing achieved by the application of the cosmetic to the face.
A: Very uniform.
B: Slightly uniform.
C: Not very uniform.

### (Finishing Performance Test 2: Dullness in External Appearance of Molded Product)

Overall evaluation of the finishing performance was carried out by a panel of five experts. Regarding the evaluation method, the powdery solid cosmetic was filled in a medium-sized dish container, and the appearance of the surface of the cosmetic was visually observed to perform evaluation according to the following standard in terms of whether or not dullness was found.
A: No dullness.
B: Slight dullness.
C: High level of dullness.

### <<Method of Producing Powdery Solid Cosmetic: Wet Molding Method>>

### Example 1

A mixture was prepared by mixing predetermined amounts of the powder part and the oil part shown in Table 1 under heat using a Henschel mixer. To the mixture, 10% ethanol in water in the same amount as the mixture and a predetermined amount of the water part described in Table 1 were added, and the resulting mixture was stirred to prepare a slurry solution. The resulting slurry solution was filled into a nearly square-shaped (about 3 cm×about 4 cm), medium-sized resin dish container having a depth of 5 mm, and then dried after removing water and ethanol, to obtain a powdery solid cosmetic of Example 1.

### Examples 2 to 6 and Comparative Examples 1 to 7

Powdery solid cosmetics of Examples 2 to 6 and Comparative Examples 1 to 7 were obtained in the same manner as in Example 1 except that different formulae shown in Table 1 were used.

TABLE 1

| | | | | | | | | | | Com- | Com- | Com- | Com- | Com- | Com- | Com- |
|---|---|---|---|---|---|---|---|---|---|---|---|---|---|---|---|---|
| | | | Example | Example | Example | Example | Example | Example | Example | parative | parative | parative | parative | parative | parative | parative |
| Part | Component | Component name | 1 | 2 | 3 | 4 | 5 | 6 | 7 | Example 1 | Example 2 | Example 3 | Example 4 | Example 5 | Example 6 | Example 7 |
| Pow- | (c) Powder | Carboxydecyl trisiloxane- | 7.2% | 7.2% | 7.2% | 7.2% | 7.2% | 7.1% | 7.1% | 9.2% | 9.2% | 9.2% | 7.2% | 7.2% | 9.1% | 9.1% |
| der | | treated talc | | | | | | | | | | | | | | |
| | | Synthetic phlogopite iron | 11.1% | 11.1% | 11.1% | 11.1% | 11.1% | 11.1% | 11.1% | 11.1% | 11.1% | 11.1% | 11.1% | 11.1% | 11.1% | 11.1% |
| | | Boron nitride | 6.0% | 6.0% | 6.0% | 6.0% | 6.0% | 6.0% | 6.0% | 6.0% | 6.0% | 6.0% | 6.0% | 6.0% | 6.0% | 6.0% |
| | | Dimethicone-treated | 20.0% | 20.0% | 20.0% | 20.0% | 20.0% | 20.0% | 20.0% | 20.0% | 20.0% | 20.0% | 20.0% | 20.0% | 20.0% | 20.0% |
| | | synthetic phlogopite iron | | | | | | | | | | | | | | |
| | | Magnesium myristate | 0.10% | 0.10% | 0.10% | 0.10% | 0.10% | 0.10% | 0.10% | 0.10% | 0.10% | 0.10% | 0.10% | 0.10% | 0.10% | 0.10% |
| | | (HDI/trimethylol | 3.8% | 3.8% | 3.8% | 3.8% | 3.8% | 3.8% | 3.8% | 3.8% | 3.8% | 3.8% | 3.8% | 3.8% | 3.8% | 3.8% |
| | | hexyllactone) | | | | | | | | | | | | | | |
| | | crosspolymer | | | | | | | | | | | | | | |
| | | (Diphenyl | 8.8% | 8.8% | 8.8% | 8.8% | 8.8% | 8.8% | 8.8% | 8.8% | 8.8% | 8.8% | 8.8% | 8.8% | 8.8% | 8.8% |
| | | dimethicone/vinyl | | | | | | | | | | | | | | |
| | | diphenyl | | | | | | | | | | | | | | |
| | | dimethicone/silsesquioxa | | | | | | | | | | | | | | |
| | | ne) crosspolymer | | | | | | | | | | | | | | |
| | | Nylon-12 | 4.4% | 4.4% | 4.4% | 4.4% | 4.4% | 4.4% | 4.4% | 4.4% | 4.4% | 4.4% | 4.4% | 4.4% | 4.4% | 4.4% |
| | | (IPDI/poly(1,4- | 2.0% | 2.0% | 2.0% | 2.0% | 2.0% | 2.0% | 2.0% | 2.0% | 2.0% | 2.0% | 2.0% | 2.0% | 2.0% | 2.0% |
| | | butanediol)-14) | | | | | | | | | | | | | | |
| | | crosspolymer | | | | | | | | | | | | | | |
| | | Barium sulfate | 1.0% | 1.0% | 1.0% | 1.0% | 1.0% | 1.0% | 1.0% | 1.0% | 1.0% | 1.0% | 1.0% | 1.0% | 1.0% | 1.0% |
| | | Dextrin palmitate-treated | 4.0% | 4.0% | 4.0% | 4.0% | 4.0% | 4.0% | 4.0% | 4.0% | 4.0% | 4.0% | 4.0% | 4.0% | 4.0% | 4.0% |
| | | zinc oxide | | | | | | | | | | | | | | |
| | | Stearic acid-treated | 3.5% | 3.5% | 3.5% | 3.5% | 3.5% | 3.5% | 3.5% | 3.5% | 3.5% | 3.5% | 3.5% | 3.5% | 3.5% | 3.5% |
| | | particulate titanium oxide | | | | | | | | | | | | | | |
| | | Stearic acid-treated | 10.0% | 10.0% | 10.0% | 10.0% | 10.0% | 10.0% | 10.0% | 10.0% | 10.0% | 10.0% | 10.0% | 10.0% | 10.0% | 10.0% |
| | | particulate titanium oxide | | | | | | | | | | | | | | |
| | | Dimethicone-treated iron | 0.08% | 0.08% | 0.08% | 0.08% | 0.08% | 0.08% | 0.08% | 0.08% | 0.08% | 0.08% | 0.08% | 0.08% | 0.08% | 0.08% |
| | | oxide | | | | | | | | | | | | | | |
| | | Silicone-treated iron oxide | 1.4% | 1.4% | 1.4% | 1.4% | 1.4% | 1.4% | 1.4% | 1.4% | 1.4% | 1.4% | 1.4% | 1.4% | 1.4% | 1.4% |
| | | (yellow) | | | | | | | | | | | | | | |
| | | Silicone-treated iron oxide | 0.1% | 0.1% | 0.1% | 0.1% | 0.1% | 0.1% | 0.1% | 0.1% | 0.1% | 0.1% | 0.1% | 0.1% | 0.1% | 0.1% |
| | | (black) | | | | | | | | | | | | | | |
| | | Mica titanium | 1.7% | 1.7% | 1.7% | 1.7% | 1.7% | 1.7% | 1.7% | 1.7% | 1.7% | 1.7% | 1.7% | 1.7% | 1.7% | 1.7% |
| water | Microbicide | Chlorphenesin | 0.2% | 0.2% | 0.2% | 0.2% | 0.2% | 0.2% | 0.2% | 0.2% | 0.2% | 0.2% | 0.2% | 0.2% | 0.2% | 0.2% |
| | (a) Water- | Glycylglycine (logP value: | 0.05% | | | 0.05% | 0.05% | 0.05% | 0.05% | 0.05% | | | 0.05% | 0.05% | 0.05% | 0.05% |
| | soluble agent | −2.3) | | | | | | | | | | | | | | |
| | | Potassium 4- | | 0.05% | | | | | | | 0.05% | | | | | |
| | | methoxysalicylate (ionic | | | | | | | | | | | | | | |
| | | water-soluble agent) | | | | | | | | | | | | | | |
| | | Tranexamic acid (logP | | | 0.05% | | | | | | | 0.05% | | | | |
| | | value: −2.0) | | | | | | | | | | | | | | |
| | | Dimethylpyrazolyl | | | | | | 0.05% | | | | | | | | 0.05% |
| | | dimethylpyrimidine | | | | | | | | | | | | | | |
| | | hydrochloride (logP value: | | | | | | | | | | | | | | |
| | | 0.8) | | | | | | | | | | | | | | |
| | | Hydroxyethyl | | | | | | | 0.05% | | | | | | 0.05% | |
| | | imidazolidinone (logP | | | | | | | | | | | | | | |
| | | value: −1.4) | | | | | | | | | | | | | | |
| Oil | Antiseptic | Phenoxyethanol | 0.5% | 0.5% | 0.5% | 0.5% | 0.5% | 0.5% | 0.5% | 0.5% | 0.5% | 0.5% | 0.5% | 0.5% | 0.5% | 0.5% |
| | Deodorant | Ethylhexyl glycerin | 0.2% | 0.2% | 0.2% | 0.2% | 0.2% | 0.2% | 0.2% | 0.2% | 0.2% | 0.2% | 0.2% | 0.2% | 0.2% | 0.2% |
| | Moisturizing | Glycerin | 0.5% | 0.5% | 0.5% | 0.5% | 0.5% | 0.5% | 0.5% | 0.5% | 0.5% | 0.5% | 2.5% | 0.5% | 0.5% | 0.5% |
| | agent | | | | | | | | | | | | | | | |
| | (b) Usability- | Dipropylene glycol (logP | 2.0% | 2.0% | 2.0% | | | 2.0% | 2.0% | | | | | | | |
| | improving | value: −0.5) | | | | | | | | | | | | | | |
| | agent | | | | | | | | | | | | | | | |
| | (polyhydric | 1,3-Butylene glycol (logP | | | | 2.0% | | | | | | | | | | |
| | alcohol) | value: −0.4) | | | | | | | | | | | | | | |
| | Polyether | Polyethylene glycol 1500 | | | | | | | | | | | | 2.0% | | |
| | | (Mixture of PEG-6 and | | | | | | | | | | | | | | |
| | | PEG-32: number average | | | | | | | | | | | | | | |
| | | molecular weight, about | | | | | | | | | | | | | | |
| | | 1500) | | | | | | | | | | | | | | |
| | (b) Usability- | Polyethylene glycol 400 | | | | | 2.0% | | | | | | | | | |
| | improving | (PEG-8: number average | | | | | | | | | | | | | | |
| | agent | molecular weight, about | | | | | | | | | | | | | | |
| | (polyether) | 400) | | | | | | | | | | | | | | |
| | Oil | Ethylhexyl | 5.0% | 5.0% | 5.0% | 5.0% | 5.0% | 5.0% | 5.0% | 5.0% | 5.0% | 5.0% | 5.0% | 5.0% | 5.0% | 5.0% |
| | | methoxycinnamate | | | | | | | | | | | | | | |
| | | Dimethicone (6cs) | 2.0% | 2.0% | 2.0% | 2.0% | 2.0% | 2.0% | 2.0% | 2.0% | 2.0% | 2.0% | 2.0% | 2.0% | 2.0% | 2.0% |
| | | Macadamia nut oil | 1.8% | 1.8% | 1.8% | 1.8% | 1.8% | 1.8% | 1.8% | 1.8% | 1.8% | 1.8% | 1.8% | 1.8% | 1.8% | 1.8% |
| | | polyglyceryl-6 esters | | | | | | | | | | | | | | |
| | | behenate | | | | | | | | | | | | | | |
| | | Glyceryl | 2.5% | 2.5% | 2.5% | 2.5% | 2.5% | 2.5% | 2.5% | 2.5% | 2.5% | 2.5% | 2.5% | 2.5% | 2.5% | 2.5% |
| | | tri(caprylate/caprate) | | | | | | | | | | | | | | |
| | | Tocopherol | 0.02% | 0.02% | 0.02% | 0.02% | 0.02% | 0.02% | 0.02% | 0.02% | 0.02% | 0.02% | 0.02% | 0.02% | 0.02% | 0.02% |
| Total | | | 100% | 100% | 100% | 100% | 100% | 100% | 100% | 100% | 100% | 100% | 100% | 100% | 100% | 100% |
| Eval- | Usability test | Removability of the | A | A | B | B | B | A | A | C | C | C | C | C | C | C |
| uation | 1 | cosmetic | | | | | | | | | | | | | | |
| | Usability test | Applicability of the | A | A | B | B | B | A | A | C | C | C | C | C | C | C |
| | 2 | cosmetic to the skin | | | | | | | | | | | | | | |
| | Finishing | Uniformity of finishing | A | A | B | B | B | A | A | C | C | C | C | B | C | C |
| | Performance | | | | | | | | | | | | | | | |
| | Test 1 | | | | | | | | | | | | | | | |
| | Finishing Performance | appearance of the molded | A | A | B | B | B | A | A | C | C | C | C | B | C | C |
| | Test 2 | Dullness in the external | | | | | | | | | | | | | | |
| | | product | | | | | | | | | | | | | | |

### <Results>

As is evident from Table 1, the cosmetics of Examples 1 to 7, each of which comprises a prescribed usability-improving agent, were found to show improved usability compared to the cosmetics of Comparative Examples 1 to 7, each of which does not comprise the predetermined usability-improving agent. Further, the cosmetics of Examples 1 to 7 were found to show favorable results also for the finishing performance.

### REFERENCE SIGNS LIST

11 Water-soluble agent
12, 22 Powder particle
13, 23 Dispersion medium
21 Mixture containing a water-soluble agent and a usability-improving agent

1. A powdery solid cosmetic comprising:
(a) a water-soluble agent;
(b) a usability-improving agent; and
(c) a powder;
wherein the (b) usability-improving agent comprises at least one selected from the group consisting of a polyhydric alcohol having a log P value of −1.5 or more, and a polyether having a number average molecular weight of 1000 or less.

2. The cosmetic according to claim 1, wherein the (a) water-soluble agent comprises at least one selected from the group consisting of an ionic water-soluble agent, and a nonionic water-soluble agent having a log P value of less than 2.0.

3. The cosmetic according to claim 1, wherein the content of the (a) water-soluble agent is 0.01% by mass or more with respect to the total amount of the cosmetic.

4. The cosmetic according to claim 1, wherein the (a) water-soluble agent comprises at least one selected from the group consisting of glycylglycine, potassium 4-methoxysalicylate, tranexamic acid or a salt thereof, dimethylpyrazolyl dimethylpyrimidine hydrochloride, hydroxyethyl imidazolidinone, and niacinamide.

5. The cosmetic according to claim 1, wherein the content of the (b) usability-improving agent is 0.1% by mass or more with respect to the total amount of the cosmetic.

6. The cosmetic according to claim 1, wherein the (b) usability-improving agent comprises at least one selected from the group consisting of dipropylene glycol, 1,3-butylene glycol, and a polyethylene glycol having a number average molecular weight of 1000 or less.

7. A method of producing the cosmetic according to claim 1, wherein wet molding is carried out.

8. The production method according to claim 7, wherein the wet molding uses water as a dispersion medium.

9. The method of producing the cosmetic according to claim 11, wherein dry molding is carried out.
